(19) United States
(12) Patent Application Publication
(10) Pub. No.: US 2007/0010655 A1
(43) Pub. Date: Jan. 11, 2007

(54) Stationary phases and a purification process using the stationary phases

(76) Inventors: Firoz D. Antia, RAHWAY, NJ (US); Russell Boyd, Monroe, NJ (US); Jimmy O. DaSilva, Oak Ridge (GB); Kent E. Golden, Fanwood, NJ (US); Joseph NtiGyabaah, Somerset, NJ (US); Christopher J. Welch, Cranbury, NJ (US)

Correspondence Address: MERCK AND CO., INC, P O BOX 2000, RAHWAY, NJ, 07065-0907, US

(21) Appl. No.: 10/569,155

(22) PCT Filed: Sep. 1, 2004
(86) PCT No.: PCT/US04/28657

§ 371 Date: Feb. 21, 2006

Related U.S. Application Data
(60) Provisional application No. 60/500,624, filed on Sep. 5, 2003.

(51) Int. Cl. A61K 38/11; C07K 7/16

(52) U.S. Cl. 530/315; 530/359

(57) ABSTRACT

This invention relates to a novel stationary phase of Formula I and a method for purifying a peptide or lipopeptide in liquid chromatography using select stationary phases, including the stationary phases of Formula I to improve the resolution and/or productivity of the purification. This chromatographic method can be used for either an analytical or preparative scale purification.

### BACKGROUND OF THE INVENTION

Lipopeptides, such as Pneumocandin B₀, are often the product of a fermentation process. During such a fermentation process, many closely related analogues are produced along with the desired product. Liquid chromatography systems are frequently used to purify the crude fermentation product. A liquid chromatography system usually consists of a stationary phase and a mobile phase. For purification of a peptide or lipopeptide, the stationary phase can be silica gel, alumina or other materials, and the mobile phase can be a single solvent or a mixture of solvents, which includes organic solvents and water.

Silica gel chromatography and other types of liquid chromatography are useful for separating these analogues. However, in practice, the separation of certain closely related analogues from the desired product is often un-satisfactory, because of poor chromatographic resolution, i.e. overlap of chromatographic peaks. To achieve the desired purity of the main product at a reasonable yield requires restricting the amount of material (often referred to as feed or column load) loaded onto the column per run, which limits the productivity of the operation.

The chromatographic purification of Pneumocandin B₀ has historically been difficult owing to poor chromatographic resolution. The chromatography utilizes a mobile phase consisting of a mixture of solvents, specifically ethyl acetate (EtOAc), methanol (MeOH) and water, on a silica gel column. In the past, separation of key impurities, such as that of Pneumocandins B₅ and E₀ from Pneumocandin B₀, was difficult owing to poor chromatographic resolution. Some analogs were only partially resolved from the main product peak using preparative conditions. To achieve the desired product purity with this limited resolution required that the purification step be run with low column loading, which limited productivity.

Pneumocandin B₀, with a molecular weight of 1065 Daltons, is a natural product and serves as an intermediate in the production of Caspofungin acetate (Cancidas®). Pneumocandin B₀ is produced as a secondary metabolite by fermentation of the fungus Glarea lozoyensis. See U.S. Pat. Nos. 5,194,377 and 5,202,309. The structures of Pneumocandin B₀ and three of the key analog impurities, all comprised of a cyclic hexapeptide coupled with dimethylmyristate side chain, are shown in Table 1.

TABLE 1
Pneumocandin B₀ and three of its analogs

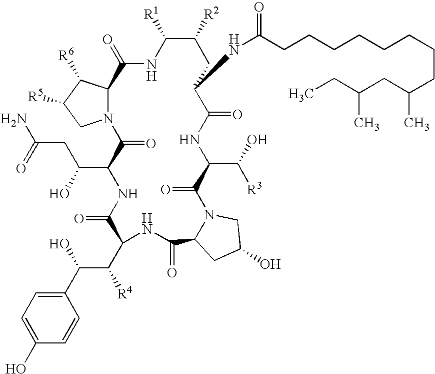

| Compound | R¹ | R² | R³ | R⁴ | R⁵ | R⁶ |
|---|---|---|---|---|---|---|
| Pneumocandin B₀ | OH | OH | Me | OH | H | OH |
| Pneumocandin B₅ | OH | H | Me | OH | H | OH |
| Pneumocandin C₀ | OH | OH | Me | OH | OH | H |
| Pneumocandin E₀ | OH | OH | Me | OH | H | H |

Silica gel chromatography exploits the subtle variations in binding affinity of the hydroxy-rich cyclic hexapeptide core of the desired product and the analog impurities, including Pneumocandins B₅, C₀, and E₀, to effect a separation. In the silica gel HPLC purification, Pneumocandins B₅ and E₀, two of the key analog impurities co-produced in the fermentation of Pneumocandin B₀, elute very closely to Pneumocandin B₀. Therefore, to meet the target impurity levels in the purified material for these and similar analogs, the quantity of crude Pneumocandin B₀ that can be loaded onto the column is limited. As a result, significant efforts have been made to improve the resolution of key impurities. For instance, the ternary ethyl acetate-methanol-water mobile phase has been balanced to optimize resolution between Pneumocandin B₀ and key analog impurities. D. J. Roush, F. D. Antia, K. E. Göklen J. Chromatography A, 827 (1998) 373-389. Additionally, the use of a mobile phase modifier has been demonstrated to enhance the resolution and selectivity between Pneumocandin B₀ and key analog impurities. See J. Nti-Gyabaah, et al., “Large-scale purification of pneumocandin B₀, a precursor for CANCIDAS”, PREP-2003, 16th International Symposium, Exhibit and Workshops on Preparative/Process Chromatography, San Francisco, Calif., Wednesday, Jul. 2, 2003 or U.S. Provisional Application No. 60/422,356 filed Oct. 30, 2002.

### SUMMARY OF THE INVENTION

This invention relates to a novel stationary phase of Formula I and a method for the purification of a peptide or a lipopeptide by using a liquid chromatography system with select stationary phases, including the stationary phases of Formula I and a mobile phase, to improve the selectivity and/or productivity of the purification.

### BRIEF DESCRIPTION OF THE DRAWINGS

FIG. 1:

Chromatogram (absorbance 278 nm vs. time) of silica gel chromatographic purification of crude Pneumocandin B₀.

FIG. 2:

Chromatogram (absorbance 278 nm vs. time) of silica gel chromatographic purification of crude Pneumocandin B₀ with a proline-modified mobile phase.

FIG. 3:

Chromatogram (absorbance 278 nm v. time) of aminopropyl-silica gel chromatographic purification of crude Pneumocandin B₀.

FIG. 4:

Chromatogram (absorbance 278 nm vs. time) of Amide-80-silica gel chromatographic purification of crude Pneumocandin B₀.

FIG. 5:

Capacity factors of Pneumocandin B₀ and related analogs on silica gel, silica gel-proline modified, aminopropyl silica gel and Amide-80-silica gel.

FIG. 6:

Chromatogram (absorbance 278 nm vs. column volumes) for a silica gel chromatography of crude Pneumocandin B₀ using a proline-modified mobile phase 88:9:7 ethyl acetate:methanol:water eluent with 0.12 g/L proline (presaturated column) and 75:17:8 ethyl acetate:methanol:water as feed solvent mixture with 1.5 g/L proline in the feed.

FIG. 7:

Chromatogram (absorbance 278 nm vs. column volumes) for an Amide-80-silica gel chromatography of crude Pneumocandin B₀ using phase 88:9:7 ethyl acetate:methanol:water eluent and 75:20:8 ethyl acetate:methanol:water as feed solvent mixture.

FIG. 8:

Chromatogram (absorbance 278 nm vs. column volumes) for N-L-prolyl-3-aminopropyl silica gel chromatography of crude Pneumocandin B₀ using phase 88:9:7 ethyl acetate:methanol:water eluent and 75:20:8 ethyl acetate:methanol:water as feed solvent mixture.

FIG. 9:

Chromatogram (absorbance 278 nm vs. column volumes) for N-methylcarbamoyl-3-aminopropyl silica gel chromatography of crude Pneumocandin B₀ using 88:9:7 ethyl acetate:methanol:water eluent and 75:20:8 ethyl acetate:methanol:water as feed solvent mixture.

FIG. 10:

Chromatogram (absorbance 278 nm vs. column volumes) for N-β-alaninamidopropyl silica gel chromatography of crude Pneumocandin B₀ using 88:9:7 ethyl acetate:methanol:water eluent and 75:20:8 ethyl acetate:methanol:water as feed solvent mixture.

### DETAILED DESCRIPTION OF THE INVENTION

A stationary phase of Formula I: 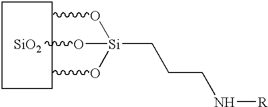
wherein
R is:

a) —(CH₂)ₙCONH₂, or

b) —COOR¹;
n is: 1 to 4; and
R¹ is: C₁-C₂ alkyl.

An embodiment of the stationary phases of Formula I are:

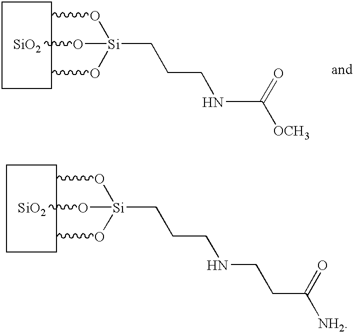

Additional stationary phases that have been useful in the purification are: Amide-80 (manufactured by Tosoh Biosep LLC., Japan),

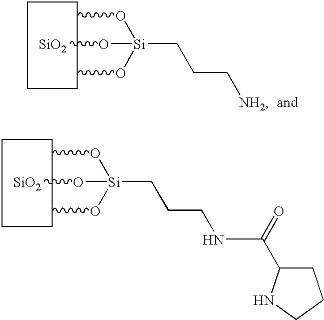

manufactured by Eka Chemicals of Sweden
described in U.S. Pat. No. 6,342,160.

A method for the purification of a peptide or a lipopeptide by using a liquid chromatography system with a stationary phase selected from the group consisting of: the stationary phases of Formula I, as recited above, Amide-80, 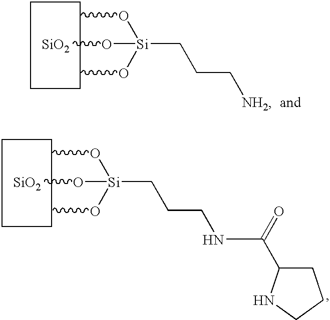
and a mobile phase, to improve the selectivity and/or productivity of the purification is disclosed.

Additional stationary phases may be useful in the purification of certain peptides and lipopeptides, which fall within the scope of formula Ia as defined below: 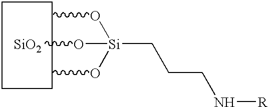
wherein
R is:

a) H,

b) N-acetyl-D-Asparginyl,

c) D-Glutaninyl,

d) L-Prolinyl,

e) Iso-L-Glutaminyl,

f) —(CH₂)ₙNH₂,

g) —(CH₂)ₙCONH₂,

h) —CO(CH₂)ₙCO₂H,

i) —CONH₂,

j) CONHR¹,

k) —COOR¹, or

l) —COR²;
n is: 1 to 4;
R¹ is C₁-C₆ alkyl, unsubstituted or substituted with one, two or three substituents selected from Cl, F, Br or I; and
R² is C₁-C₆ alkyl, unsubstituted or substituted with one, two or three substituents selected from Cl, F, Br or I, aryl, wherein aryl is defined as phenyl or napthyl, unsubstituted or substituted with one, two or three substituents selected from Cl, F, Br, I, or nitro.

Examples of lipopeptides, for which this purification process is useful, are echinocandin derivatives, such as Pneumocandin B₀, Caspofungin, Cilofungin and Micafungin as well as Anidulafungin and Daptomycin, and particularly the natural product precursors cof Caspofungin, Micafungin, Cilofungin, Anidulafungin and Daptomycin. The natural product/fermentation product precursor for Caspofungin is Pneumocandin B₀. Caspofungin acetate (CANCIDAS®) is a semisynthetic lipopeptide echinocandin B derivative currently being sold in the US as an antifungal agent for intravenous administration. Anidulafungin is a semisynthetic lipopeptide echinocandin B derivative under development by Eli Lilly/Versicor as an antifungal agent for intravenous administration. Anidulafungin is disclosed in U.S. Pat. Nos. 5,965,525 and 6,384,013, hereby incorporated by reference. Cilofungin is an echinocandin lipopeptide disclosed by Eli Lilly in U.S. Pat. No. 4,293,489 for use as an antifungal agent, hereby incorporated by reference. Micafungin (FUNGARD™) is an echinocandin-like lipopeptide under development by Fujisawa, as an antifungal agent for intravenous administration. Micafungin is disclosed in U.S. Pat. No. 6,107,458 hereby incorporated by reference. Daptomycin (CIDECIN™) is a semisynthetic lipopeptide derivative under development by Cubist as an antibacterial agent. Daptomycin is disclosed by Eli Lilly in U.S. Pat. No. 4,537,717 hereby incorporated by reference.

A liquid chromatography system employs a mobile phase and a stationary phase. The mobile phase is a solvent system comprising one or more solvents, the composition of which is either constant throughout the purification process, or a gradient, where the solvent composition is changed over time during the purification process. The mobile phase solvents include, but are not limited to, water, methanol, ethanol, isopropanol, hexane, heptane, ethyl acetate, isopropyl acetate, acetonitrile, methyl t-butyl ether (MTBE) and methylene chloride. The stationary phase is selected from the group consisting of: the stationary phases of Formula I: 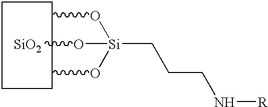
wherein R is: —(CH₂)ₙCONH₂, or —COOR¹; n is: 1 to 4; and R¹ is: C₁-C₂ alkyl;
Tosoh Amide 80,

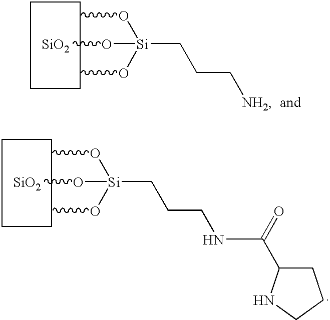

The instant invention provides a chromatographic purification method for a peptide or lipopeptide, which employs a silica gel amino- or amide-containing stationary phase. A column volume (hereinafter referred to as cv) is defined as the volume of solvent needed to traverse the column. Column load refers to the amount of material (crude lipopeptide or peptide) that is applied to the column is a single injection cycle. Column load may also be referred to as column feed or feed load.

The examples provided herein are intended to assist in a further understanding of the invention. Particular materials, employed species and conditions are intended to be further illustrative of the invention and not limitative of the reasonable scope thereof.

### EXAMPLE 1

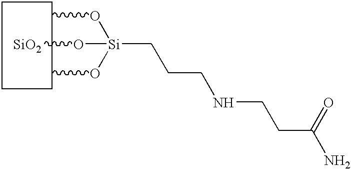

### Preparation of N-β-alaninamidopropyl silica

Kromasil amino silica (5 g, 10μ, 100 Å) was placed in a 100 mL round bottom flask, to which was added 25 mL of dichloromethane. Following complete wetting of the stationary phase, aided by gentle swirling, a solution of acrylamide containing 25-30 ppm cupric ion as a free radical inhibitor (14 mMoles) in 25 mL of dichloromethane was added, and the mixture was rotated overnight on a rotary evaporator apparatus at room temperature and without applied vacuum. The following morning, the mixture was filtered on a sintered glass funnel, washed three times with 30 mL of 20% methanol in dichloromethane, taken up in a slurry with 2-propanol, and packed into a 4.6 mm id×25 cm length HPLC column for evaluation. Residual stationary phase taken from the column packer reservoir was dried overnight under high vacuum, then submitted for combustion analysis (C 6.3%; N 1.8%).

### EXAMPLE 2

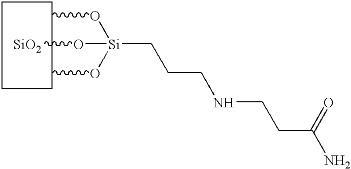

### In Situ Preparation of N-β-alaninamidopropyl silica

A commercial amino silica column (Chromegabond Amine; 5μ particle size; 60 Å pore size; 4.6 mm column i.d.; 25 cm column length) was flushed with dichloromethane at a flow rate of 2 mL/min for a period of 30 minutes. Then a 10 M solution of acrylamide in acetonitrile was circulated through the aminopropyl silica HPLC column at a flow rate of 2 mL/ml n, the effluent from the column being directed to the pump inlet reservoir so as to allow reagent recirculation. Flow of the reagent solution was continued for a period of 4 hours, whereupon the column was washed with a solution of first dichloromethane at 2 mL/min for 20 minutes, then 20% methanol in dichloromethane at 2 mL/min for 20 minutes.

### EXAMPLE 3

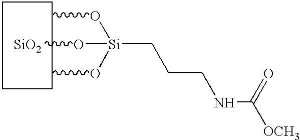

### Preparation of N-methylcarbamoyl-3aminopropyl silica

Kromasil amino silica (5 g, 10μ, 100 Å) was placed in a 100 mL round bottom flask, to which was added 25 mL of dichloromethane. Following complete wetting of the stationary phase, aided by gentle swirling, a solution of methyl chloroformate (10 mMoles) and triethylamine (10 mMoles, 1.0 eq) in 25 mL of dichloromethane was then added, and the mixture was rotated overnight on a rotary evaporator apparatus at room temperature and without applied vacuum. The following morning, the mixture was filtered on a sintered glass funnel, washed three times with 30 mL of 20% methanol in dichloromethane, taken up in a slurry with 2-propanol, and packed into a 4.6 mm id×25 cm length HPLC column for evaluation. Residual stationary phase taken from the column packer reservoir was dried overnight under high vacuum, then submitted for combustion analysis (C 6.0%; N 1.4%).

### EXAMPLE 4

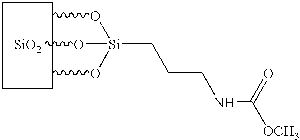

### In Situ Preparation of N-methylcarbamoyl-3-aminopropyl silica

A commercial amino silica column (Chromegabond Amine; 5μ particle size; 60 Å pore size; 4.6 mm column i.d.; 25 cm column length) was flushed with dichloromethane at a flow rate of 2 mL/min for a period of 30 minutes. A 1 M solution of the methyl chloroformate in dichloromethane containing one equivalent of triethylamine was then passed through the column at a flow rate of 2 mL/min. The column was then flushed with dichloromethane for 10 minutes at 2 mL/min, followed by flushing with 20% methanol in dichloromethane for 20 minutes at 2 mL/min.

### EXAMPLE 5

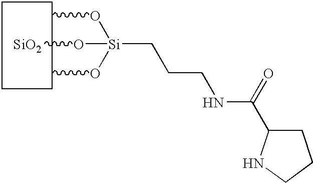

### Preparation of N-L-prolyl-3-aminopropyl silica

Kromasil amino silica (5 g, 10μ, 10 Å) was placed in a 100 mL round bottom flask, to which was added 25 mL of dichloromethane. Following complete wetting of stationary phase, aided by gentle swirling, a solution of Boc-L-Pro (4.7 mMoles) and 1-ethyl-3-(3-dimethylaminopropyl)carbodiimide hydrochloride (EDC, 4.7 mMoles, 1.0 eq) in 25 mL of dichloromethane was then added, and the mixture was rotated overnight on a rotary evaporator apparatus at room temperature and without applied vacuum. The following morning, the mixture was filtered on a sintered glass funnel, washed three times with 30 mL of 20% methanol in dichloromethane, and dried under high vacuum. Following drying, 45 mL of a 35% solution of trifluoroacetic acid (TFA) in dichloromethane was added, and the resulting slurry was shaken at room temperature for 45 minutes. The slurry was then filtered, and the recovered stationary phase was washed three times with 30 mL 20% methanol in dichloromethane, then with 30 mL of a solution of 10% Hunig's base in dichloromethane, then taken up in a slurry with 2-propanol, and packed into a 4.6 mm id×25 cm length HPLC column for evaluation. Residual stationary phase taken from the column packer reservoir was dried overnight under high vacuum, then submitted for combustion analysis (C 7.5%; N 2.0%).

### EXAMPLE 6

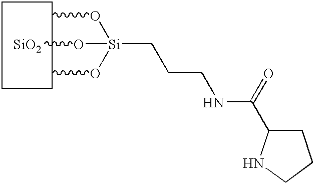

### In Situ Preparation of N-L-prolyl-3-aminopropyl silica

A commercial amino silica column (Chromegabond Amine; 5μ particle size; 60 Å pore size; 4.6 mm column i.d.; 25 cm column length) was flushed with dichloromethane at a flow rate of 2 mL/min for a period of 30 minutes. A 1 M solution of the Boc-L-Pro in acetonitrile containing one equivalent of 1-ethyl-3-(3-dimethylaminopropyl)carbodiimide hydrochloride was then passed through the column at a flow rate of 2 mL/min for 50 minutes. The column was flushed with dichloromethane for 10 minutes at 2 mL/min, followed by flushing with 20% methanol in dichloromethane for 20 minutes at 2 mL/min. Removal of the Boc protecting group was performed in situ by first flushing the column with dichloromethane at 5 mL/min for 10 minutes, followed by a solution of 4% trifluoroacetic acid in dichloromethane at 5 mL/min for 40 minutes, which is then flushed out with dichloromethane at 5 mL/min for 20 minutes. Then, a solution of 0.5% triethylamine in dichloromethane is pumped through the column at 5 mL/min for 40 minutes, followed by dichloromethane at 5 mL/min for 20 minutes.

### EXAMPLE 7

### Comparison of HPLC of Pneumocandin B₀ on Different Stationary Phases at Low Loadings

Analytical scale HPLC columns, 250 mm long×4.6 mm id, were used to compare the retention and selectivity characteristics obtained with different stationary phases when using an analytical loading of Pneumocandin B₀ and its analogues. The different stationary phases included: (1) regular silica, and (2) aminopropyl silica (obtained from Eka Chemicals (Bohus, Sweden)), and (3) Amide-80 (an amide moiety bonded to silica, obtained from Tosoh Biosep LLC. (Japan)). All these packings had a 10 μm particle size and 120 Å pore diameter. The regular silica was run as a simple liquid chromatography system, and using a mobile phase modified with L-proline, resulting in saturation of the silica with proline. See J. Nti-Gyabaah, et al., “Large-scale purification of pneumocandin B₀, a precursor for CANCIDAS”, PREP-2003, 16th International Symposium, Exhibit and Workshops on Preparative/Process Chromatography, San Francisco, Calif., Wednesday, Jul. 2, 2003 or U.S. Provisional Application No. 60/422,356 filed Oct. 30, 2002.

The ternary mobile phase and the feed diluent (84/9/7 v/v/v ethyl acetate, methanol and water) were made using HPLC grade solvents from Fisher Scientific (Pittsburg, Pa., USA). L-proline used for the proline elution silica run was obtained from Ajinomoto (Japan). The L-proline was dissolved in the ternary mobile phase at 0.12 g/L. The feed was prepared by blending pure Pn B₀ (crude Pn B₀ that had been purified by the standard silica gel method) with aliquots of solutions containing Pn B₅, Pn C₀ and Pn E₀, so that these analogs were each present at roughly 10% the concentration of Pn B₀, which was present at roughly 1 g/L. The Pn C₀-enriched solution was obtained by selecting tailcut fractions from an injection of crude Pn B₀ on silica gel using the standard silica gel method. The Pn B₅- and Pn E₀-enriched solutions were obtained by selecting forecut fractions from an injection of Pn B₀ on silica gel using the proline elution method, and then further purifying those cuts by a reversed-phase method similar to that employed for analytical analysis of Pn B₀ (described later in this example) but with higher feed loading. An Agilent HP-1100 HPLC system (Waldbronn, Germany) with diode array detector was used for the HPLC runs, as well as for fraction analysis; a wavelength of 278 nm was used for detection.

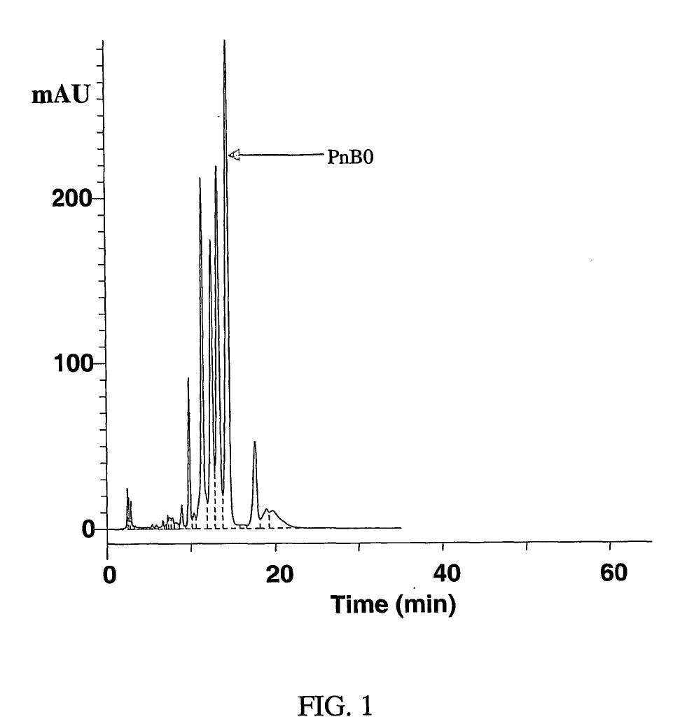

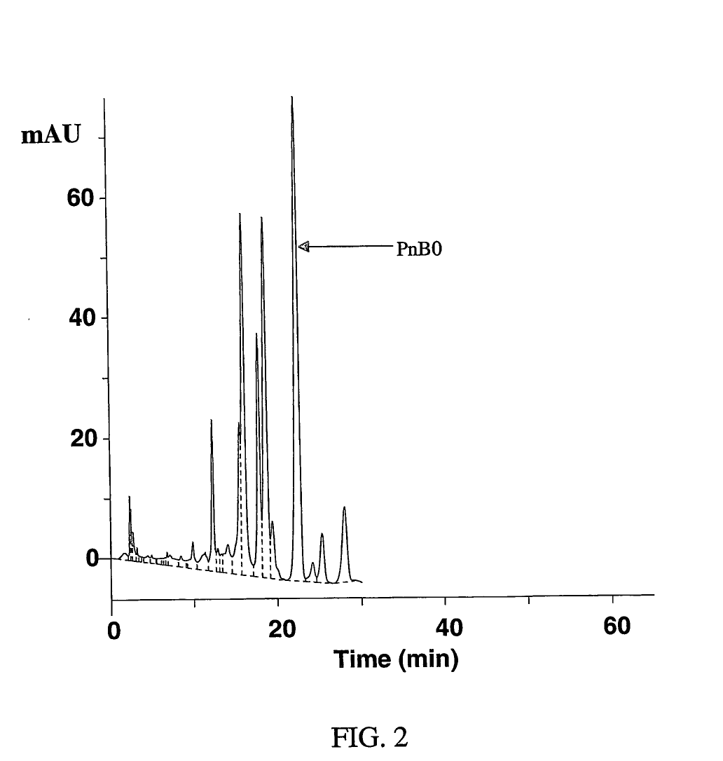

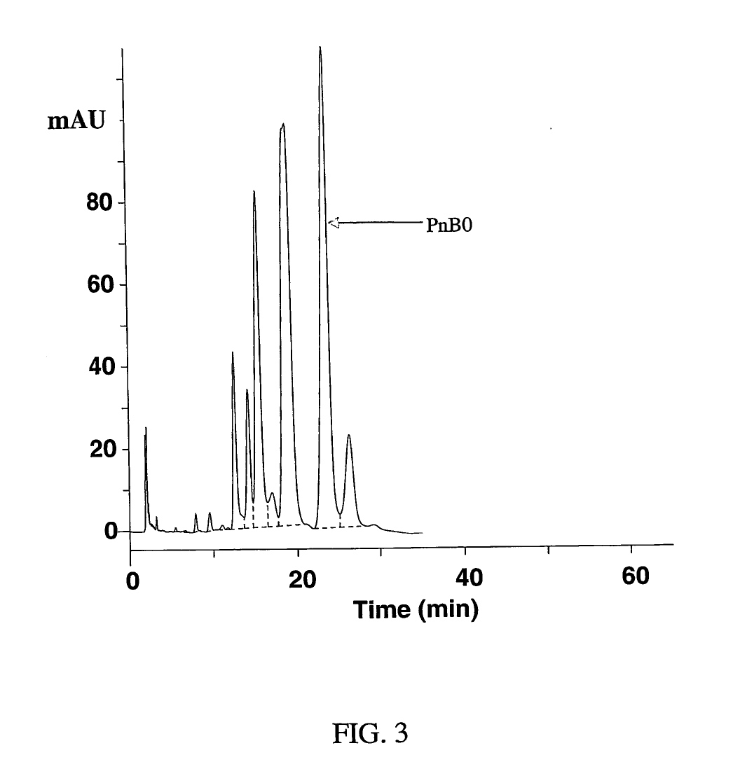

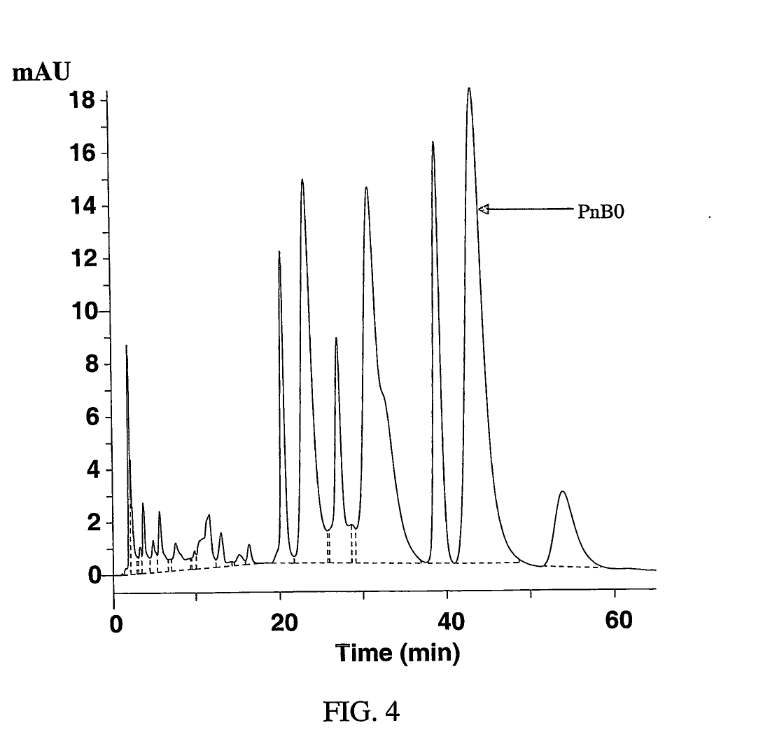

For each injection, 10 μL of the 1 g/L feed solution was injected. The mobile phase flow rate was 1.1 mL/min. For the run using the proline-modified mobile phase and regular silica, L-proline was dissolved in the mobile phase at ˜0.12 g/L and ˜480 mL of the solution was then pumped through the silica column (to in situ coat the silica with proline). The L-proline containing solution was used as eluent for that run. Chromatograms obtained from the runs are shown in FIGS. 1, 2, 3, and 4.

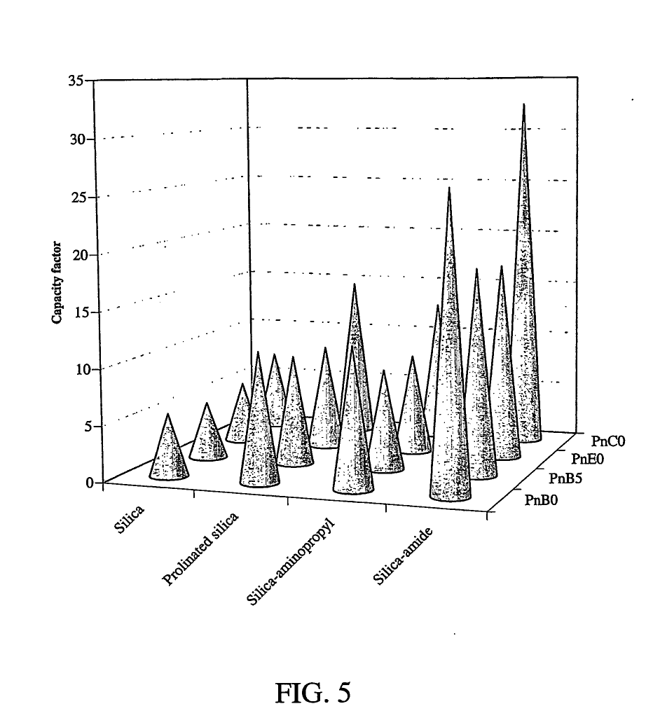

For each run, the peaks were identified by collecting fractions and analyzing them by normal and reversed phase HPLC using methods described in U.S. Provisional Application No. 60/422,356 filed Oct. 30, 2002 (incorporated by reference) or J. Nti-Gyabaah, et al., “Large-scale purification of pneumocandin B₀, a precursor for CANCIDAS”, PREP-2003, 16th International Symposium, Exhibit and Workshops on Preparative/Process Chromatography, San Francisco, Calif., Wednesday, Jul. 2, 2003. The retention times of pneumocandins B₀, E₀, B₅ and C₀ were then used to calculate the capacity factors, from which the selectivities between Pn B₀ and the three analogs were obtained. The results are shown in FIGS. 1-5 and Table 2.

TABLE 2
Selectivities between Pneumocandin B₀ and analogs

Selectivity (Relative to Pn B₀)

| | Pn E₀ | Pn B₅ | Pn C₀ | FIGS. |
|---|---|---|---|---|
| Bare silica | 1.03 | 1.05 | 1.27 | 1 |
| “Prolinated” silica* | 1.22 | 1.25 | 1.26 | 2 |
| Aminopropyl-silica | 1.67 | 1.66 | 1.06 | 3 |
| Amide-80 silica | 1.46 | 1.42 | 1.25 | 4 |

*silica gel chromatography using a proline-modified mobile phase

### EXAMPLE 8

### Purification of Pneumocandin B₀ Using Amide-80 Silica as the Stationary Phase

Amide-80 bonded phase was obtained as a 250 mm long×4.6 mm id column (10 am particle size, 8 nm pore size, spherical) from Tosoh Biosep LLC. A separation using the same feed material on a bare silica column equilibrated with proline-modified mobile phase was performed as a control, using a W. R. Grace/Davison silica gel Grade-631 column (250 mm long×4.6 mm id, 16-2(Tm particle size, 60 Å pore size, irregular) supplied by Princeton Chromatography. The mobile phase composition for both runs was 88/917 v/v/v ethyl acetate/methanol/water (e/m/w); the proline-modified mobile phase also contained 0.12 g/L of L-proline. All solvents were HPLC grade from Fisher Scientific. Proline was obtained from Ajinomoto (Japan).

A partially purified preparation of Pneumocandin B₀ (Pn B₀ crude) with a purity of 61.9% was used to prepare the column feed. The methods for preparing Pn B₀ crude are given in U.S. Pat. Nos. 5,202,309, 5,194,377 and 6,610,822. For the Amide-80 run, the feed solution was prepared by dissolving Pn B₀ crude into a 75/20/8 v/v/v mixture of ethyl acetate, methanol and water; the concentration of Pneumocandin B₀ in the feed solution was ˜45 g/L. For the control run, a 75/17/8 v/v/v ethyl acetate/methanol/water feed solvent mixture was employed, with 1.5 g/L proline, and ˜45 g/L Pn B₀.

Before use, the columns were flushed with 10 column volume (cv) of methanol and equilibrated with 10 cv of the mobile phase. For each injection, 1.2 mL of the feed solution was injected (representing ˜15 g/L bed). The mobile phase flow rate was ˜0.5 ml/min. For each run, fractions were collected and analyzed to assess the quality of the separation. Each injection was repeated to ensure a consistent elution profile. For the run employing proline-modified mobile phase, ˜480 mL of the proline-containing mobile phase was pumped through the bare silica column prior to the first injection.

Analytical Methods:

For each run, fractions were collected and analyzed by reversed and normal phase HPLC. The samples were analyzed on an Agilent HP-1100 analytical HPLC system. The reversed phase HPLC was used to quantify most species, except for Pn C₀ which co-elutes with Pn B₀ in the reversed phase method. The normal phase HPLC assay was used to determine Pn C₀.

Measurement of Pn C₀

The amount of Pn C₀ was determined by normal phase Pn B₀ assay, an isocratic HPLC method, which employs a YMC silica column (SL12S05-2546WT) with a particle size of 5 μm and a pore size of 120 Å. The column was 250×4.6 mm i.d. and maintained at 25° C. Elution was isocratic with 84/9/7 ethyl acetate/methanol/water eluent. Flow rate was 1.2 mL/min, and detection was by UV absorbance of 278 nm. Product samples required no special preparation prior to injection.

Measurement of Pn B₀ and Other Species

The reversed phase HPLC assay is used to measure Pn B₀/Pn C₀ and the other species, including Pn E₀ and Pn B₅. The reversed phase assay uses a gradient HPLC method with an YMC J'Sphere column (JM08S04-2546WT), particle size of 4 μm and pore size of 80 Å. The column was 250×4.6 mm i.d. and maintained at 30° C. The two mobile phases used were 0.1% phosphoric acid (A) and acetonitrile (B). The elution gradient started at 60% A and 40% B and ramped to 1% A and 99% B over 45 minutes at 1.5 mL/min, with UV detection at 220 nm. Prior to analysis, samples were blown dry under nitrogen and re-dissolved in methanol to the original concentration.

Results

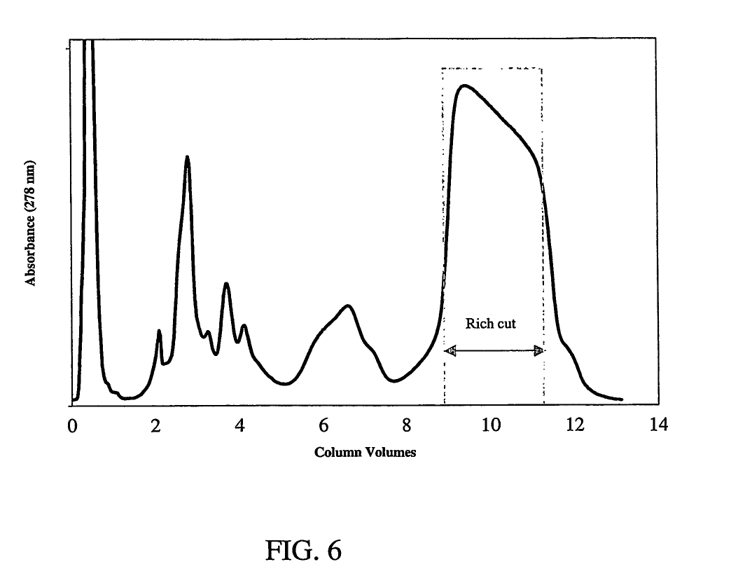

A typical chromatogram obtained using the proline-modified mobile phase on bare silica gel is shown in FIG. 6. The Pn B₀ elutes as a large rich cut as indicated in the figure, with excellent resolution from Pn E₀ and Pn B₅, which elute in the preceding peak.

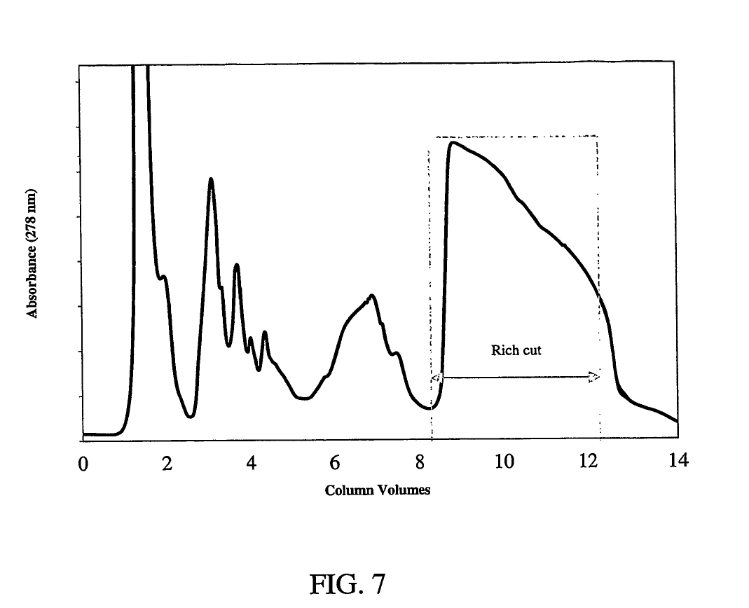

A chromatogram obtained from a run on the Amide-80 column is shown in FIG. 7. No proline or other modifier was employed in either the mobile phase or feed solvent for this run. A slightly higher level of methanol is used in the feed solvent mixture than for the control case. The retention of Pn B₀ on this solid phase was similar to that achieved for the control run with the proline-modified mobile phase (shown in FIG. 6). In fact, the elution profile appears very similar to the control system, except that the main Pn B₀ peak is broader.

Many fractions were collected from ˜6 to 14 column volumes (cv) and the fractions were analyzed by both normal and reversed phase HPLC. This analysis confirmed that analogues such as Pn E₀ and Pn B₅ are well resolved from Pn B₀; these analogues were contained in fractions representing ˜6 to 8.5 cv in FIG. 7. Results of the normal phase analysis indicated that most of Pn C₀ was resolved into the fractions representing 13 to 14 cv in FIG. 7. Therefore, the fractions representing ˜8.5 to 13 cv, shown in FIG. 7, could be combined to give a pure rich cut with yield >90%.

### EXAMPLE 9

### Purification of Pneumocandin B₀ Using N-L-prolyl-3-aminopropyl silica as the Stationary Phase

A column was prepared containing the N-L-prolyl-3-aminopropyl silica stationary phase; methods for preparing this stationary phase were presented in Examples 5 and 6. The column utilized was 250 mm long×4.6 mm id, and contained the proline-amide moiety bonded to spherical silica from ES Industries which had 5 μm particle size and 60 Å pore size. The experiment was otherwise identical to that described in Example 8, and the results may also be evaluated against the control in that example as illustrated in FIG. 6.

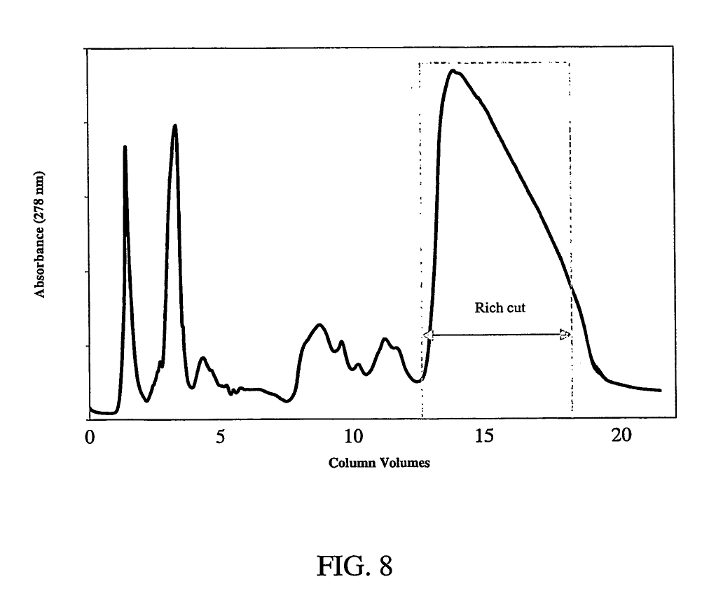

The chromatogram obtained from the run on the N-L-prolyl-3-aminopropyl silica bonded phase is shown in FIG. 8. As illustrated, despite the increased levels of methanol in the feed and the exclusion of proline from the system, the retention of Pn B₀ on this phase was similar to that achieved on bare silica using the proline-modified mobile phase (shown in FIG. 6). However, there are some key differences. First, the large solvent front peak normally observed in control system appears diminished. Another difference is that impurities that elute at the leading edge of the main Pn B₀ peak appear to be well separated from each other. Finally, there is more tailing of the Pn B₀ peak (compared to FIG. 6).

Many fractions were collected from ˜10 to 21 cv and the fractions were analyzed by both normal and reversed phase HPLC. The analytical results indicated the key early eluting impurities such as Pn B₅ and Pn E₀ were clearly resolved from Pn B₀. These impurities are mostly contained in the fraction which represented the 10-13 cv range in FIG. 8. Results of the normal phase analysis indicate most of the Pn C₀ was resolved into the fraction that represented the 13 to 14 cv range in FIG. 8. Therefore, ˜13 to 19 cv (shown in FIG. 8) could be combined to give a pure rich cut with yield >90%.

### EXAMPLE 10

### Purification of Pneumocandin B₀ Using N-methylcarbamoyl-3-aminopropyl Silica as the Stationary Phase

A column was prepared containing the N-methylcarbamoyl-3-aminopropyl silica stationary phase; methods for preparing this stationary phase were presented in Examples 3 and 4. The column in this example was 250 mm long×4.6 mm id, and contained the N-methylcarbamoyl-3-aminopropyl silica moiety bonded to spherical silica from ES Industries which had 5 μm particle size and 60 Å pore size. The experiment was otherwise identical to that described in Example 8, and the results may also be evaluated against the control in that example as illustrated in FIG. 6.

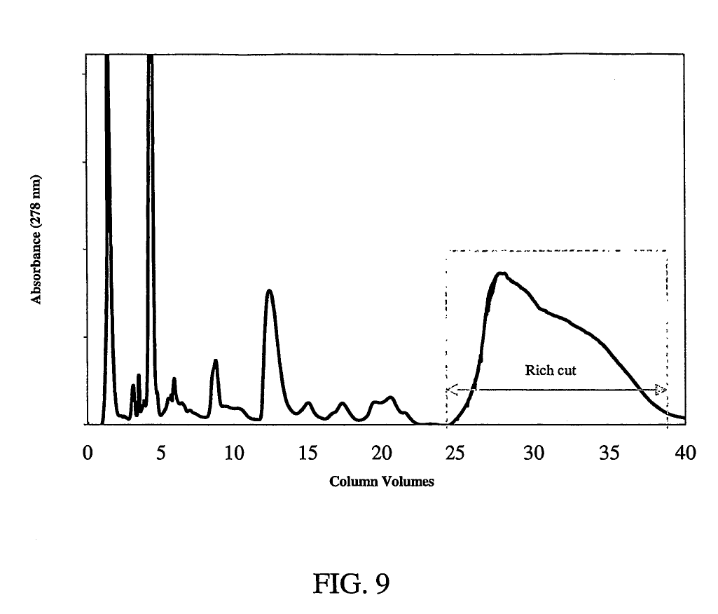

A chromatogram obtained from the run on the N-methylcarbamoyl-3-aminopropyl silica bonded phase is illustrated in FIG. 9. Despite the increased methanol in the feed and the exclusion of proline from the system, the retention of Pn B₀ on this phase was excellent (compared to FIG. 6). Many fractions were collected from ˜21 to 40 cv. The fractions were analyzed by normal and reversed phase HPLC. The fraction analysis indicated that the early eluting analogues such as Pn E₀ and Pn B₅ were well resolved from Pn B₀ and contained in the fractions representing ˜21 to 25 cv, shown in FIG. 9. Results of the normal phase analysis indicated that the resolution of Pn B₀ from later eluting analogues such as Pn C₀ was comparable to that in the control process. A ring-opened degradate, which was seen at only trace levels if at all in the previous examples, was present at levels of a few percent in the rich cuts of this run. Other than this degradate, the later fractions were very pure.

### EXAMPLE 11

### Purification of Pneumocandin B₀ Using N-β-alaninamidopropyl Silica as the Stationary Phase

A column was prepared containing the N-β-alaninamidopropyl silica stationary phase; methods for preparing this stationary phase were presented in Examples 1 and 2. The column used was 250 mm long×4.6 mm id, and contained one propylamide moiety bound to an aminopropyl moiety bonded to spherical silica from ES Industries which had 5 μm particle size and 60 Å pore size. The experiment was similar to that described in Example 8, but the stationary phase was significantly more retentive than most of the others evaluated, and so a stronger mobile phase (75/17/8 ethyl acetate/methanol/water) was needed to completely elute Pn B₀ from the column. The results may still be evaluated against the control as illustrated in FIG. 6.

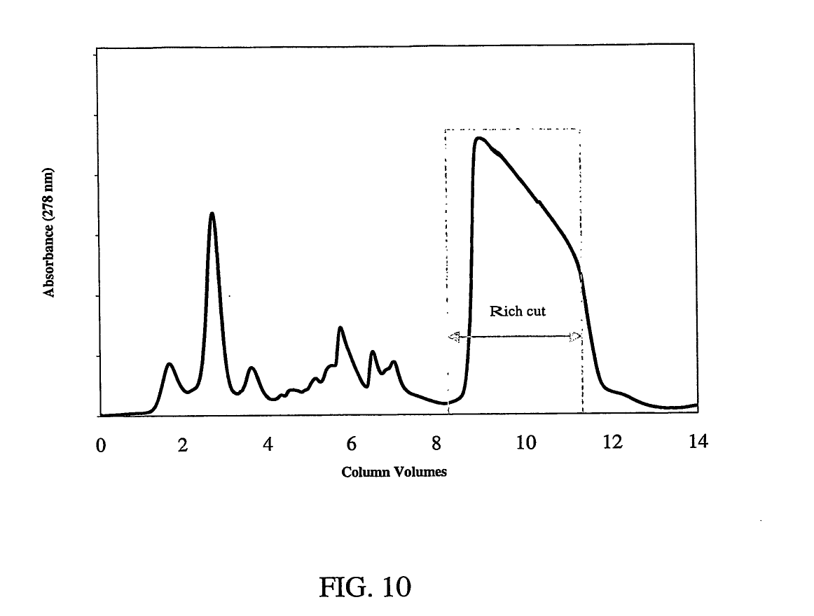

A chromatogram obtained from the run on the N-β-alaninamidopropyl silica bonded phase is shown in FIG. 10. It is important to point out that the large solvent front peak normally observed in the proline elution method system appears diminished. Another difference is that the key impurities that elute at the leading edge of the main Pn B₀ peak are well resolved.

Several fractions were collected from ˜6 to 13 cv. The fractions were analyzed by both normal and reversed phase HPLC. The analytical results indicated that the early eluting analogues such as Pn E₀ and Pn B₅ were clearly resolved from Pn B₀. These impurities were mostly contained in the fractions that represent ˜6 to 8 cv. Results of the normal phase analysis indicate that most of the Pn C₀ was resolved into the fractions that represent 11 to 13 cv. Fractions that represent ˜8 to 11 cv (shown in FIG. 10) could be combined to give a pure rich cut with >90% yield. A low level of the ring-opened degradate was detected in the rich cut fractions. This is a potentially superior stationary phase, since its retentivity allows use of a mobile phase for elution which is very similar in solvent strength to that of the feed solvent mixture, which results in very stable chromatographic performance.

What is claimed is:

1. A stationary phase of Formula I: 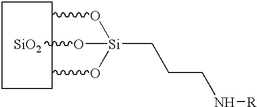
wherein
R is:
c) —(CH₂)ₙCONH₂, or
d) —COOR¹;
n is: 1 to 4; and
R¹ is: C₁-C₂ alkyl.

2. The stationary phase of Formula I, as recited in claim 1, selected from the group consisting of:

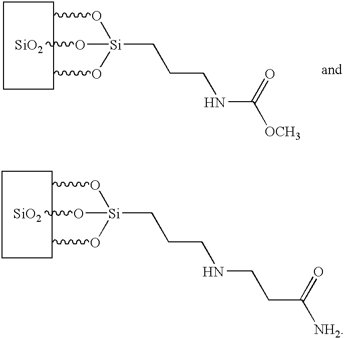

3. A method for the purification of a peptide or a lipopeptide by using a liquid chromatography system with a stationary phase selected from the group consisting of: the stationary phase of Formula I, as recited in claim 1, Tosoh Amide 80, 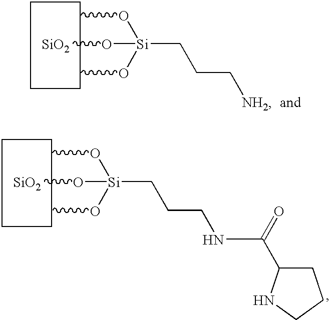
and a mobile phase, to improve the selectivity and/or productivity of the purification.

4. The method as recited in claim 3, wherein the mobile phase is a solvent system comprising one or more solvents.

5. The method as recited in claim 4, wherein the solvents in the solvent system are selected from the group consisting of: water, methanol, ethanol, isopropanol, hexane, heptane, ethyl acetate, isopropyl acetate, acetonitrile, methyl t-butyl ether (MTBE) and methylene chloride.

6. The method as recited in claim 4, wherein the liquid chromatography system is for the purification of a peptide.

7. The method as recited in claim 4, wherein the liquid chromatography system is for the purification of a lipopeptide.

8. The method as recited in claim 7, wherein the lipopeptide is a fermentation product precursor of caspofungin, micafungin, cilofungin, andulifungin and daptomycin.

9. The method as recited in claim 8, wherein the lipopeptide is the precusor of caspofungin, pneumocandin B₀.

10. The method as recited in claim 9, wherein the mobile phase is a solvent system comprising water, methanol, and ethyl acetate.

11. The method as recited in claim 6, wherein the peptide is oxytocin or bradykinin.

12. A method of purifying Pneumocandin B₀ with a liquid chromatography system comprising a stationary phase selected from: Tosoh amide 80, 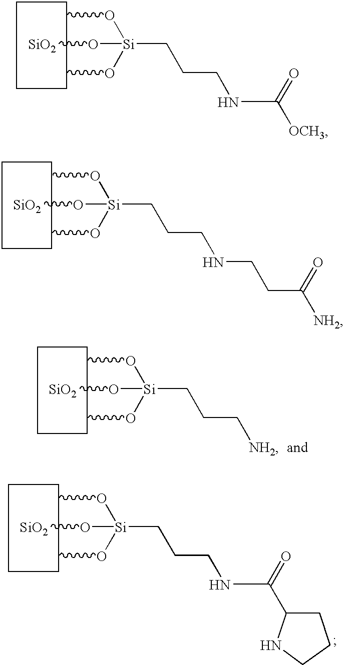
and a mobile phase to improve the selectivity and/or productivity of the purification.

13. The method as recited in claim 12, wherein the mobile phase comprises a solvent system, wherein the solvents in the solvent system are selected from the group consisting of: water, methanol, ethanol, isopropanol, hexane, heptane, ethyl acetate, isopropyl acetate, acetonitrile, methyl t-butyl ether (MTBE) and methylene chloride.

14. The method as recited in claim 13, wherein the solvent system is a mixture of ethyl acetate, methanol and water.
